(19) United States
(12) Patent Application Publication
(10) Pub. No.: US 2020/0015691 A1
(43) Pub. Date: Jan. 16, 2020

(54) Apparatus and Method for A Physiological Parameter Monitoring Device

(71) Applicant: Paul Tobias, JR., Mitchell, IN (US)

(72) Inventor: Paul Tobias, JR., Mitchell, IN (US)

(21) Appl. No.: 16/035,496

(22) Filed: Jul. 13, 2018

(51) Int. Cl. A61B 5/024; A61B 5/021; A61B 5/00

(52) U.S. Cl. CPC A61B 5/02438; A61B 5/021; A61B 5/681; A61B 5/02405; G04G 17/04; A61B 2560/045; A61B 5/746; A61B 5/6898; A61B 5/7207

(57) ABSTRACT

A monitoring device is presented. The monitoring device has a circuit-board enclosure having a cradle containing a removable battery, wherein the cradle is in engagement with a transceiver module for contacting a wrist of a person. A wrist strap is described for attaching the wearable device to the wrist of a user. The monitoring device has a sensor for sensing change in a blood-related parameter and a memory device for storing the blood-related parameters. The memory device is connected to the circuit-board and a display so that the display can show the result of the determined blood-related parameter information.

### FIELD OF THE INVENTION

The present invention relates to improvements in devices designed to monitor physiological parameters.

### BACKGROUND

Monitoring physiological parameters associated with a person provides useful health information. Conventional physiological parameter monitoring is designed to be used by trained professionals in a clinical setting.

Lately, it has become more common to see mobile devices, such as watches, monitoring cardiovascular activity of its users. These mobile devices have proved beneficial for many. The deaf and blind community's needs have not been addressed by these current mobile devices. There is a need in for deaf and blind communities to have access to a mobile device that can be easily useable by them.

### BRIEF DESCRIPTION OF THE DRAWINGS

FIG. 1 is a general circuitry for the monitoring device.

FIG. 2 is a view of the communications circuitry of the monitoring device.

FIG. 3 is the top and side view of the monitoring device.

FIG. 4 is the bottom view of the monitoring device.

### DETAILED DESCRIPTION

Reference will now be made in detail to the present preferred embodiments, examples of which are illustrated in the accompanying drawings. All terms in the plural shall also be taken as singular and vice-versa. Further, any reference to he shall also be applicable to she and vice-versa.

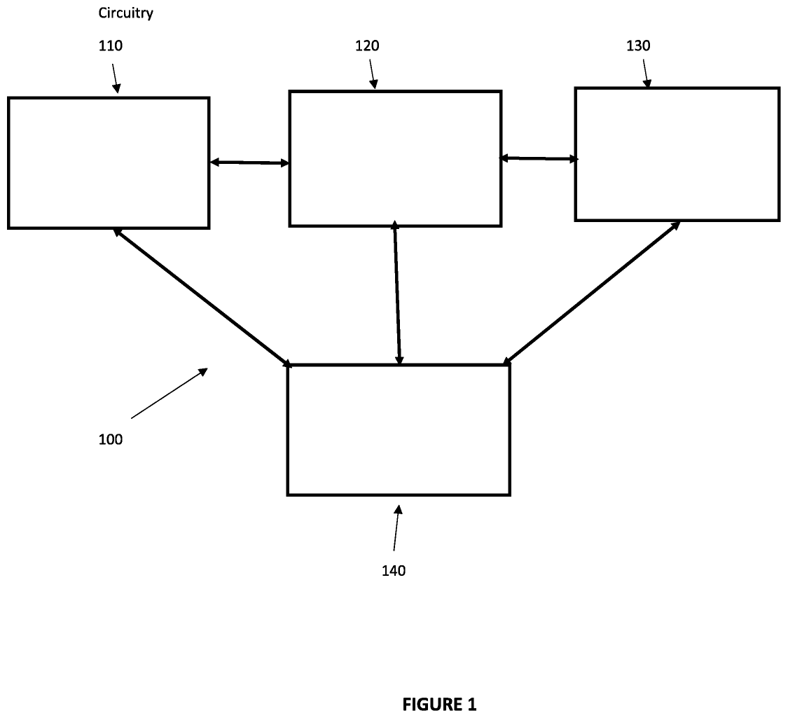

FIG. 1 provides a block diagram of the general circuitry blocks 100 and the interconnection thereof. The preferred embodiment thus provides an analog circuit block 110, a digital controller block 120, a communications block 130 and a power supply and power management block 140. Essentially, the electrodes pick up ECG (electrocardiograph) signals from the heart. The ECG signal is then conditioned to remove undesirable attributes, i.e., noise, from the signal. A second set of electrodes pick up the oxygen level of the blood. The analog Signal is converted to a digital signal and then digitally processed under software algorithms.

In the preferred embodiment, the device can store 24 hours of real time data, although longer and/or shorter storing periods are contemplated by this application. The amount of storage and time frame of the storage is dependent on the memory accessible for storing the monitoring results. The details of the electronic circuitry are well known in the art and are not further described herein.

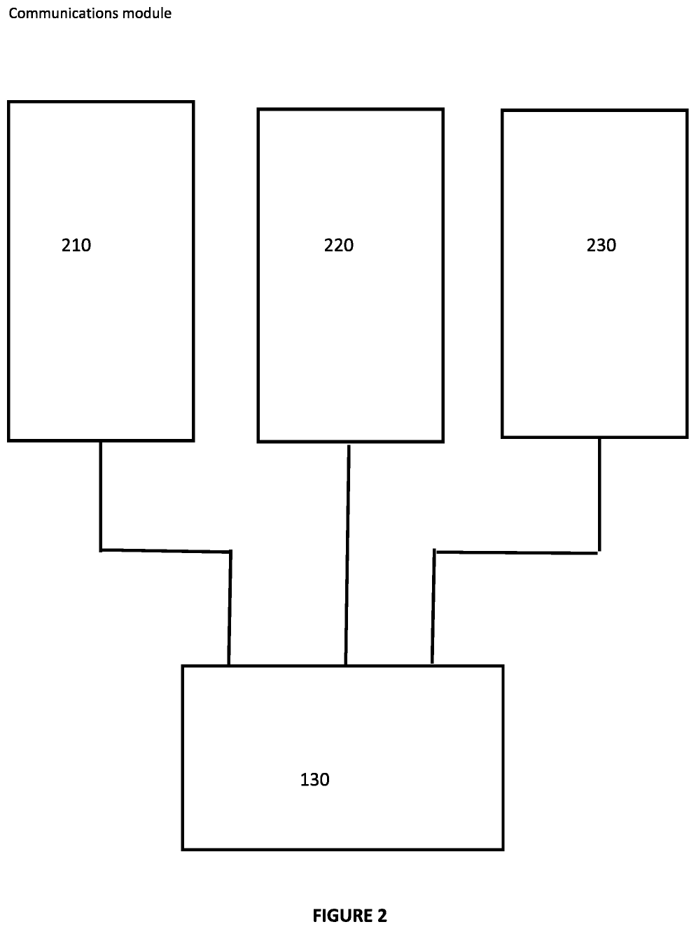

Now referring to FIG. 2, a block diagram of the communications block 130 is presented which is interconnected with different external communication methods. It is desirable and useful to be able to either store the acquired data internally within the device, externally or to transmit it to external devices. Therefore, it is contemplated that conventional, preferably high-speed communications with external devices is an aspect of the present invention; it is contemplated that at least three types of transceivers accomplish this objective, each transceiver having different attributes and utility. For direct connection to a personal computer for further review, study and analysis of the data, high speed wired links are contemplated in the form of the direct connect USB 2.0 port 210.

For ambulatory data transfer, wireless links are contemplated 220. For example, connection to a wireless communications device, e.g., a Bluetooth wireless device, LTE/5G cellular connectivity, WiFi, etc., may be provided. In another embodiment, the device may have a communication with the mobile telephony network via 3G, 4G, LTE and/or 5G technology. This method provides a mechanism to alert medical personnel or others, as determined by an inputted emergency call out list, to be notified of any abnormalities or dangerous conditions detected by the device.

Alternatively, wireless USB 3.0 wireless ports are contemplated for uploading the acquired data. In addition, compatibility with certain medical instruments and notebook personal computers may include an infrared transceiver 230 as provided as part of the device. The infrared method provides a slow, but proven and direct view optical link. Additional methods of transferring data from the device will readily present themselves to those skilled in the art.

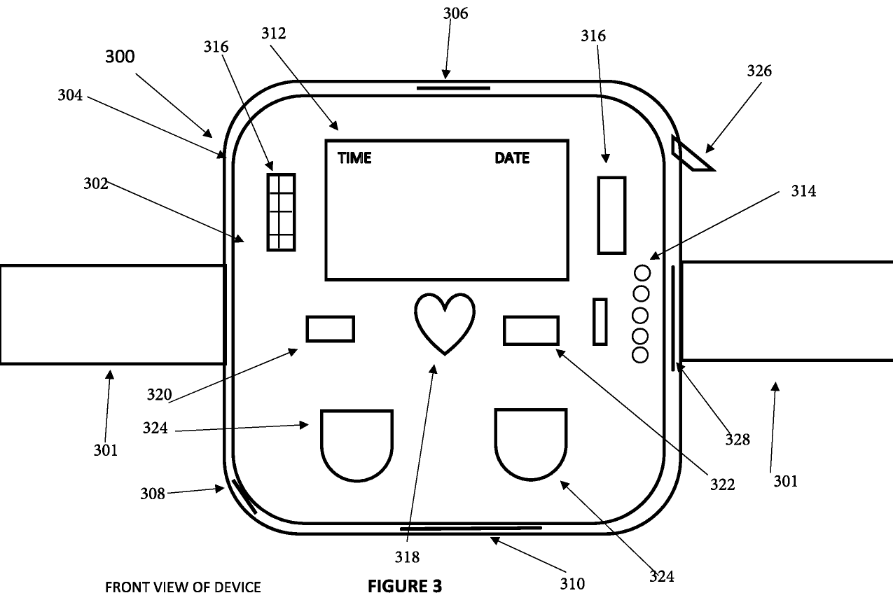

Referring now to FIG. 3, which shows the face view of the monitoring device 300. The device face 300 has a top 302 and a side 304. Attached to the monitoring device 300 are wrist straps 301 for securing the monitoring device to the user. A port 306 is on the side 304 of the device face 300, the port 304 is a charging port for connecting to the power supply 140. Port 304 can be a USB, fire connector receiver, or any other type of power charging connector. Also, on the side 304 of the device face 300 is an SD port 308 for inserting a memory card, e.g. SD card.

Further on the side 304 of the device face 300 is an oxygen monitoring port 310. The oxygen monitoring port 310 is configured such that a finger may fit into the oxygen monitoring port 310, press the blood oxygen button 320 on the top 302 of the device face 300 and the second electrodes in the oxygen monitoring port 310 sense the oxygen level of a user's blood and sends electrical signals to the general device circuitry 310 for analysis. The output of the analysis is then displayed on a display panel 312 of the top 302 of the device face 300.

Further, the lights 314 on the top 302 of the device face 300 are lit to display the status of the analysis. An alarm is sounded thru the speakers 316 on the device face 300 when the status of the analysis surpasses a status known to be of a poor condition to alert the user to seek medical help. The use of the alarm using the speakers 316 on the device face 300 allows for a blind person to be able to seek help in an emergency.

In the preferred embodiment, the lights 314 on the top 302 of the device face 300 are green indicated that the analysis has shown that the results are in the generally agreed on good range, yellow when the analysis indicates that the results are above what is generally considered to be the good range and red when the results of the analysis indicate that the results are generally considered to be in a critical condition. Though the preferred embodiment shows five indicator lights 314, one could use any number of lights to show this indication. Further, the preferred embodiment uses the color scheme of green, yellow, and red as visual indicators where one could use any color for any of the three statuses. Also, the preferred embodiment has three status levels but one could indicate any number of levels to a user.

In the preferred embodiment, the top 302 of the device face 300 has three push buttons, a power button 318, a blood oxygen button 320 and a blood glucose button 322. When a user wants to power the device on, the user would press the power button 318. If the device is currently off, then the user would press the power button 318 to turn the device off. Although not shown on the FIG. 3, braille is used to allow a person with vision problems to be able to discern the use of the buttons described in detail below.

When the user desires to have their blood glucose taken, the user removes a test strip contained in the test strip holder 324 on the top 302 of the device face 300. The user would then prick his/her finger using the finger prick 326 on the side 304 of the device face 300. The user places a sample of the blood onto the test strip, then opens the blood glucose slot 328 located on the side 304 of the device face 300. The user slides the test strip with the blood sample in the open blood glucose slot 328 on the side 304 of the device face 300.

Once the test strip has been slid into the blood glucose slot 328, the user presses the blood glucose button 322 on the top 302 of the device face 300. The circuitry receives the electronic signals for the blood sample and determines the glucose level of the blood. The results are displayed on the display 312 on the top 302 of the device face 300. The lights 328 on the top 302 of the device face 300 are lit to be the color, as described above, to indicate the status of the test results. Further, if the test results indicate a critical status, an alarm is sounded through the speakers 316 on the top 302 of the device face 300.

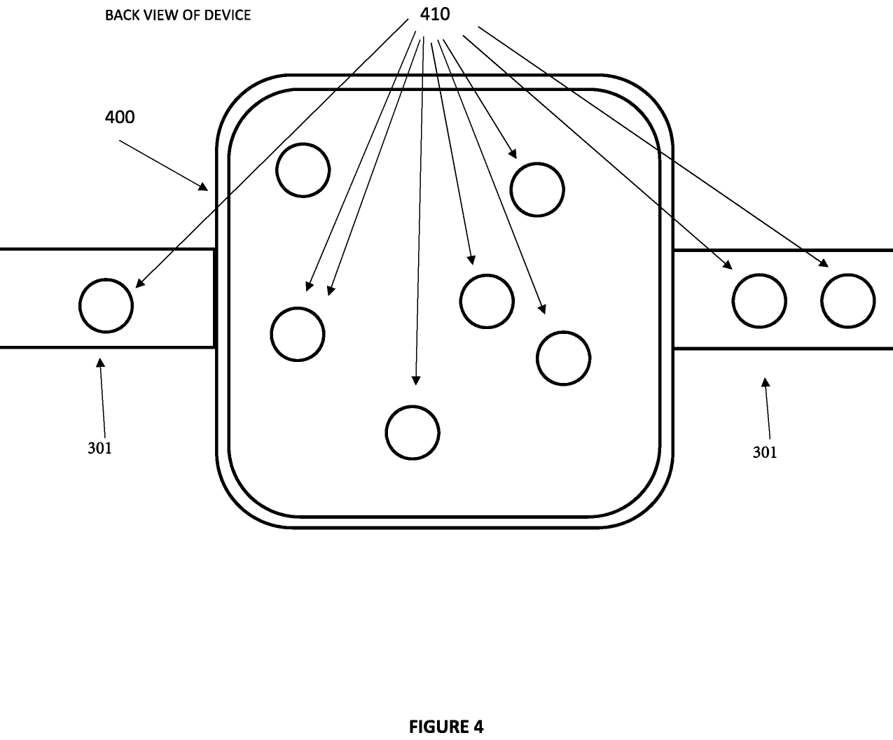

Turning now to FIG. 4, a bottom view of the monitoring device is described. The back 400 of the monitoring device has at least one sensor or electrode for testing and reporting results to the general circuitry 100 of the monitoring device. The preferred embodiment shows a plurality of sensors or electrodes, the number of sensors or electrodes could be one or more. The sensors or electrodes could be located on the back 400 of the monitoring device, on the wrist straps 301, or on both the back 400 and the wrist straps 301.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

I claim:

1. An apparatus comprising a monitoring device, the monitoring device includes:
a circuit-board enclosure having a cradle containing a removable battery, wherein the cradle is in engagement with a transceiver module for contacting a wrist of a person;
an wrist strap for encircling the wrist and removably attaching the wearable device to the wrist;
a sensor for sensing change in a blood-related parameter;
a memory device for storing the blood-related parameters, where the memory device being communicably coupled to the circuit-board and,
a display, wherein the display result of the blood-related parameter information.

2. The apparatus of claim 1, where the face of the monitoring device further has a test strip holder, wherein the test strip is for testing the sugar content of the person.

3. The apparatus of claim 1, where the monitoring device further has a finger prick device, wherein the finger prick device is for providing access to blood for analysis.

4. The apparatus of claim 1, further comprising a USB connection for charging the battery of the monitoring device.

5. The apparatus of claim 1, further comprising one or more buttons on the monitoring device and wherein a description for the one or more buttons is in braille.

6. The apparatus of claim 1, wherein the monitoring device has a speaker for alerting the person of any change in the blood-related parameters.

7. The apparatus of claim 1, further comprising indicator lights on the monitoring device; wherein the indicator lights change color to alert the person of a status of the blood-related parameters.

8. The apparatus of claim 7, wherein the color of the lights is green when an analysis of a test indicates the results are in the good range.

9. The apparatus of claim 1, further comprising an opening in the monitoring device for inserting a finger to determine blood oxygen.

10. The apparatus of claim 1, further comprising electrodes wherein the electrodes are located on the inside of the wrist strap.

11. The apparatus of claim 1, wherein the monitoring device is securely attachable to the person.

12. The apparatus of claim 1, further comprising a wireless connection for communicating blood-related parameters.

13. The apparatus of claim 1, wherein the wireless connection is Bluetooth.

14. The apparatus of claim 12, further comprising a relay transceiver for receiving signals from the Bluetooth transmitter and relaying them to a base station.
